(19) United States
(12) Patent Application Publication
(10) Pub. No.: US 2015/0037895 A1
(43) Pub. Date: Feb. 5, 2015

(54) Hydrocarbon Detector Based on Carbon Nanotubes

(71) Applicant: International Business Machines Corporation, Armonk, NY (US)

(72) Inventor: Jose M. Lobez Comeras, New York, NY (US)

(73) Assignee: International Business Machines Corporation, Armonk, NY (US)

(21) Appl. No.: 13/955,818

(22) Filed: Jul. 31, 2013

(51) Int. Cl. G01N 33/24

(52) U.S. Cl. CPC G01N 33/241
USPC 436/27; 436/143

(57) ABSTRACT

Hydrocarbon detectors having CNTs coated with an amphiphilic coating capable of sequestering an active molecule are provided. In one aspect, a hydrocarbon detection device is provided. The hydrocarbon detector device includes a plurality of CNTs dispersed in a polar solvent, wherein each of the CNTs is coated with an amphiphilic coating having molecules with a hydrophilic moiety and a hydrophobic moiety, and wherein the coating creates a hydrophobic environment proximal to a surface of each of the CNTs; and one or more hydrophobic active molecules sequestered in the hydrophobic environment proximal to the surface of each of the CNTs.

### FIELD OF THE INVENTION

The present invention relates to hydrocarbon detection and more particularly, to hydrocarbon detectors having carbon nanotubes (CNTs) coated with an amphiphilic coating capable of sequestering an active molecule in a hydrophobic environment proximal to a surface of the CNTs.

### BACKGROUND OF THE INVENTION

Hydrocarbon detection is necessary in the discovery of new underground oil reservoirs and/or for the estimation of the quality and level of depletion of an already discovered oil field. One possibility to determine the composition of underground materials is direct excavation, sample retrieval and chemical analysis using techniques such as gas chromatography. The use of this approach however has some disadvantages, the most notable of which is that sample acquisition is extremely expensive and time consuming. Further, the direct excavation approach will become even more impractical as oil reserves become scarcer and less concentrated, and when most superficial oil reserves are depleted.

Some solutions based on the injection of certain materials dispersed in a solvent into deep wells have been proposed. These more practical approaches are based on the retrieval of this dispersed material after injection into a potential well, and detection of the changes that the injected material undergoes when interacting with hydrocarbons in the underground oil well. The whole process can be done using a solvent flow scheme, in which only a very small excavation is required both to inject the active material and then to retrieve it.

A specific example of this latter approach makes use of a graphitic material that can be dispersed in water, such as oxidized carbon black coated with poly(vinyl alcohol). See, for example, Berlin et al., “Engineered nanoparticles for hydrocarbon detection in oil-field rocks,” Energy Environ. Sci., 4, 505-509 (2011) (published December 2010). This polymer coating is capable of sequestering small organic molecules that then leach when placed in contact with a hydrophobic solvent. Detection of the decrease in the concentration of this small molecule can be traced back to the amount of hydrocarbon to which the modified carbon black was exposed.

This approach however has some notable drawbacks. First, the surface area of carbon black is not very high, so the amount of coating and thus active molecule that can be incorporated is limited—which limits the sensitivity of the whole scheme. Second, the low surface area of carbon black also means that the amount of functional surface per unit weight is not very high, which limits the solubility of these systems in water, and thus their sensitivity. Third, the functionalization of other graphitic materials with a higher surface area such as carbon nanotubes (CNTs) has shown to be impractical using this approach. This is due to CNT aggregation and limited solubility caused by the cross-linking effected by the poly (vinyl alcohol), and due to low functionalization densities on the surface of the CNTs using this specific chemical transformation.

Thus, improved hydrocarbon detectors that are efficient and effective, and which solve the above-described problems would be desirable.

### SUMMARY OF THE INVENTION

The present invention provides hydrocarbon detectors having carbon nanotubes (CNTs) coated with an amphiphilic coating capable of sequestering an active molecule. In one aspect of the invention, a hydrocarbon detection device is provided. The hydrocarbon detector device includes a plurality of carbon nanotubes dispersed in a polar solvent, wherein each of the carbon nanotubes is coated with an amphiphilic coating having molecules with a hydrophilic moiety and a hydrophobic moiety, and wherein the coating creates a hydrophobic environment proximal to a surface of each of the carbon nanotubes; and one or more hydrophobic active molecules sequestered in the hydrophobic environment proximal to the surface of each of the carbon nanotubes.

In another aspect of the invention, a method of forming a hydrocarbon detection device is provided which includes the steps of: (a) contacting carbon nanotubes with a solution comprising molecules with a hydrophilic moiety and a hydrophobic moiety solubilized in a polar solvent, wherein by way of the contacting step (a) the molecules interact with surfaces of the carbon nanotubes to form an amphiphilic coating on the surfaces of the carbon nanotubes which creates a hydrophobic environment proximal to a surface of each of the carbon nanotubes; and (b) contacting the solution with one or more hydrophobic active molecules, wherein by way of the contacting step (b) the hydrophobic active molecules are sequestered in the hydrophobic environment proximal to the surface of each of the carbon nanotubes.

In yet another aspect of the invention, another method of forming a hydrocarbon detection device is provided which includes the steps of: (a) contacting carbon nanotubes with a solution comprising polymer precursors solubilized in a polar solvent, wherein by way of the contacting step (a) the polymer precursors interact with surfaces of the carbon nanotubes, modifying the surfaces of the carbon nanotubes with the polymer precursors; (b) adding monomers to the solution, wherein by way of the adding step (b), the monomers will interact with the polymer precursors on the surfaces of the carbon nanotubes to form polymers on the surfaces of the carbon nanotubes with a hydrophilic moiety and a hydrophobic moiety, and wherein the polymers form an amphiphilic coating on the surfaces of the carbon nanotubes which creates a hydrophobic environment proximal to a surface of each of the carbon nanotubes; and (c) contacting the solution with one or more hydrophobic active molecules, wherein by way of the contacting step (c) the hydrophobic active molecules become sequestered in the hydrophobic environment proximal to the surface of each of the carbon nanotubes.

In still yet another aspect of the invention, a method of hydrocarbon detection is provided which includes the steps of: preparing a solution comprising i) a plurality of carbon nanotubes dispersed in a polar solvent, wherein each of the carbon nanotubes is coated with an amphiphilic coating comprising molecules with a hydrophilic moiety and a hydrophobic moiety, and wherein the coating creates a hydrophobic environment proximal to a surface of each of the carbon nanotubes, and ii) one or more hydrophobic active molecules sequestered in the hydrophobic environment proximal to the surface of each of the carbon nanotubes wherein the active molecules are labeled with a radioactive isotope; introducing the solution into one or more oil wells such that upon any exposure to hydrocarbons in the wells one or more of the hydrophobic active molecules are transferred from the hydrophobic environment proximal to the surface of the carbon nanotubes to the hydrocarbons in the wells; collecting the solution once the solution has passed through the wells; and analyzing radioactivity levels of the solution collected from the wells

A more complete understanding of the present invention, as well as further features and advantages of the present invention, will be obtained by reference to the following detailed description and drawings.

### BRIEF DESCRIPTION OF THE DRAWINGS

FIG. 1A is a front view of a carbon nanotube (CNT) coated with an amphiphilic coating that is capable of sequestering an active molecule according to an embodiment of the present invention;

FIG. 1B is a cross-sectional view of a CNT coated with the amphiphilic coating that is capable of sequestering an active molecule according to an embodiment of the present invention;

FIG. 2 is a diagram illustrating an exemplary sensing methodology using the present amphiphilic coated CNTs according to an embodiment of the present invention;

FIG. 3 is a diagram illustrating an exemplary methodology for forming CNT-based hydrocarbon detectors which include CNTs covalently functionalized with an amphiphilic organic coating which can sequester small (hydrophobic) active molecules in the hydrophobic environment created proximal to the surface of the CNTs according to an embodiment of the present invention;

FIG. 4 is a diagram illustrating another exemplary methodology for forming the present CNT-based hydrocarbon detectors according to an embodiment of the present invention;

FIG. 5 is a diagram illustrating an amphiphilic coating on the CNTs formed from amino-hydroxamic acid (i.e., HA-CNTs) according to an embodiment of the present invention;

FIG. 6 is a diagram illustrating an amphiphilic coating on the CNTs formed from a polymer brush bearing hydroxamic acid functionalities (pHA-CNTs) according to an embodiment of the present invention;

FIG. 7A is a diagram illustrating iodomethane labeled with radioactive iodine or carbon isotopes for use as the (hydrophobic) active molecules in the CNT-based hydrocarbon detectors according to an embodiment of the present invention;

FIG. 7B is a diagram illustrating iodobenzene labeled with radioactive iodine or carbon isotopes for use as the (hydrophobic) active molecules in the CNT-based hydrocarbon detectors according to an embodiment of the present invention;

FIG. 7C is a diagram illustrating tetrachlorobiphenylene labeled with radioactive carbon isotopes for use as the (hydrophobic) active molecules in the CNT-based hydrocarbon detectors according to an embodiment of the present invention;

FIG. 8 is a diagram illustrating an exemplary methodology for detecting the presence of underground oil according to an embodiment of the present invention; and

FIG. 9 is a diagram of an apparatus configured to perform one or more of the methodologies presented herein according to an embodiment of the present invention.

### DETAILED DESCRIPTION OF PREFERRED EMBODIMENTS

Provided herein is a new scheme for hydrocarbon detection based on carbon nanotubes (CNTs) coated with an amphiphilic coating, capable of sequestering an active molecule. This amphiphilic coating renders the CNTs very soluble in water while keeping a hydrophobic environment in proximity to the surface of the CNTs. See FIGS. 1A and 1B. Specifically, FIG. 1A illustrates a front view of a CNT 102 that is surrounded by an amphiphilic coating having a (first) hydrophobic part 104 and a (second) hydrophilic part 106.

FIG. 1B is a cross-sectional diagram illustrating how the present amphiphilic coating is capable of sequestering small (low molecular weight) molecules 108, e.g., of molecular weights less than 2000 g/mol. For example, active small molecules capable of being detected via optic, electric or radioactivity measurements can be sequestered in the hydrophobic volume created (by the coating) around the CNTs. As provided above, the hydrophilic component of the coating permits CNTs to be solubilized in water. When the coated CNTs (e.g., in an aqueous solution) come into contact with a hydrophobic material, such as a liquid hydrocarbon, the active molecules can leach from the CNT coating into the hydrocarbon.

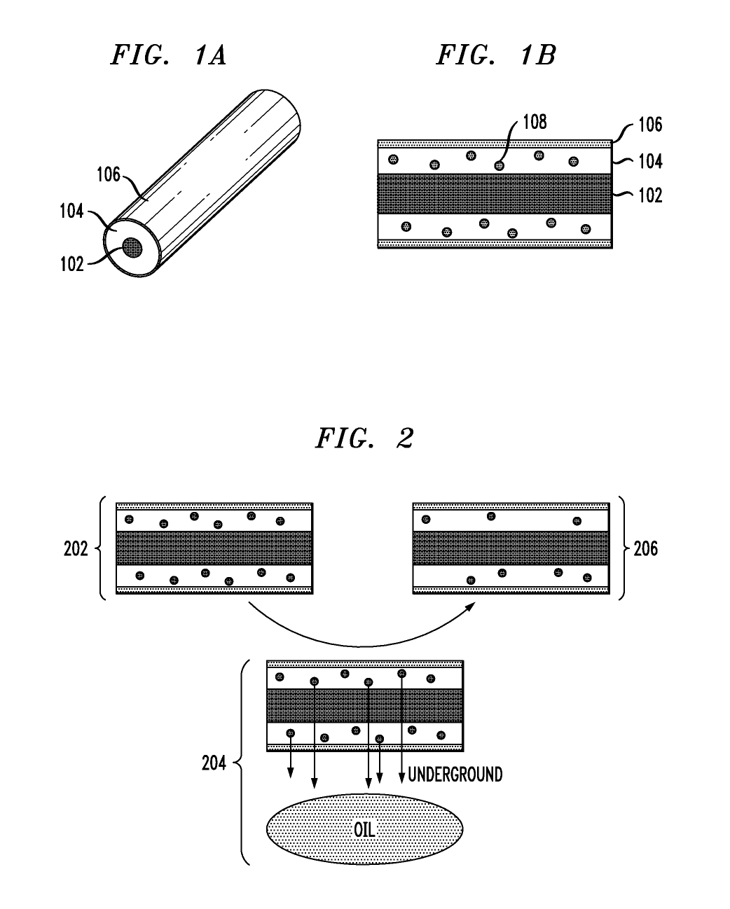

The amount of active molecules which leach from the coating is proportional to the amount of hydrophobic material to which the CNTs have been exposed. Thus, exposure of the CNTs to this hydrophobic liquid can be measured by quantification of how many of these active molecules are still present in the CNT coating. See for example, FIG. 2 which illustrates an exemplary sensing methodology using the present amphiphilic coated CNTs.

As shown in FIG. 2, in step 202, an aqueous solution of the present amphiphilic coated CNTs can be injected into the soil. By way of example only, a small well can be formed in the soil, and the amphiphilic coated CNT solution can be introduced into the well. Forming the well would require only a minimal amount of excavation.

The presence of hydrocarbons (such as oil) will cause the hydrophobic molecules to leach from the coating (into the soil). See step 204 of FIG. 2. As provided above, the amount of active molecules which leach from the coating is proportional to the amount of hydrophobic material to which the CNTs have been exposed. Thus, as per step 206 of FIG. 2, the amount of active molecules present in the CNT coating is less, post-exposure. Compare the number of active molecules from step 202 versus those in step 206. The balance of the active molecules is assumed to have leached into the soil upon exposure to the hydrophobic material (e.g., oil).

As will be described in detail below, the concentration of the active molecules in the well can be monitored to determine whether the well contains a hydrophobic material. For instance, if the active molecules contain radioactive labels, then the concentration of the active molecules in the well can be ascertained simply by measuring the radioactivity in the well. It is notable however that the present techniques can be used not only for the detection and analysis of oil fields, but also for any situation where detection of non-polar hydrophobic liquids is desired, such as in the food and chemical industry.

The present techniques have the following advantages: 1) the surface area of CNTs is much higher than that of carbon black or any other non-nanocarbon material, due to their large aspect ratio—this means that more coating and active molecule per weight unit of CNTs can be incorporated increasing the system sensitivity; 2) the CNTs are covered with a coating capable of interacting with water and thus can be incorporated into solution in concentrations much higher than other graphitic materials described in previous reports for this kind of application—this means that more coating and active molecule per volume of solvent can be incorporated increasing the system sensitivity; 3) the present CNT coating makes CNT dispersions in water extremely stable over long periods of time (e.g., dispersions can be obtained that remain stable for months), which is desired for this application since aggregation lowers the sensitivity; 4) CNTs are advantageous compared to other nanocarbon materials such as graphene or graphene nanoribbons, since CNTs are more reactive and thus more coating molecules can be incorporated onto their surface, thereby increasing solubility and their capability for sequestering active molecules.

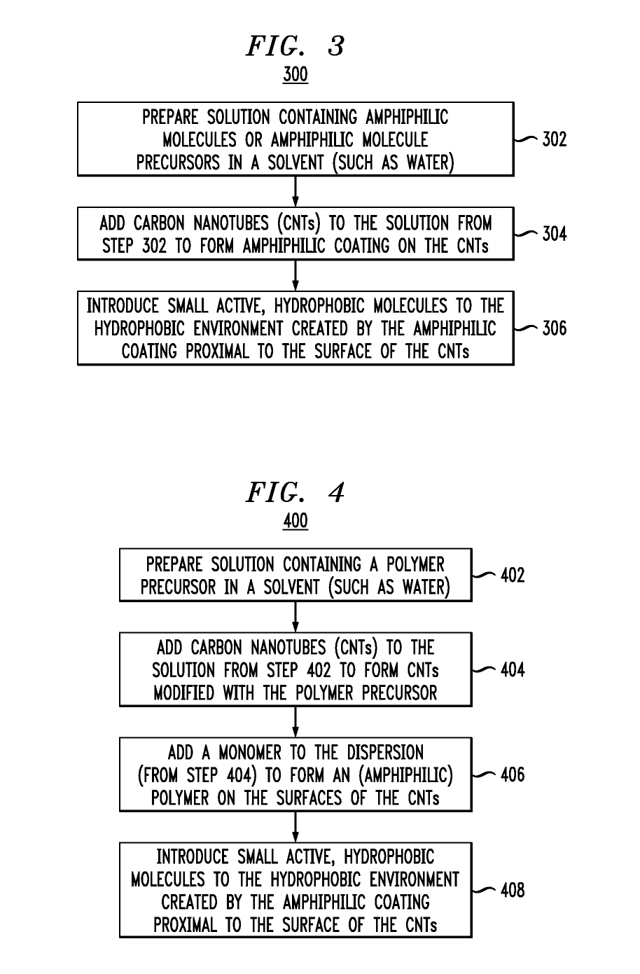

As provided above, the CNTs are covalently functionalized with an organic coating of molecules that bears both a hydrophobic moiety and a hydrophilic moiety and which can sequester small (hydrophobic) active molecules in the hydrophobic environment created proximal to the surface of the CNTs. A couple of different approaches are proposed herein for forming these CNT-based hydrocarbon detectors. The approaches differ based primarily on the steps taken to form the amphiphilic coating covalently attached to the CNTs. Namely, in one case, the coating is formed by reacting a polymer with the surface of the CNTs. This scenario is depicted in FIG. 3 (methodology 300). Alternatively, the amphiphilic coating is created on the CNTs by growing a polymer off the surface of the carbon nanotubes. This scenario is depicted in FIG. 4 (methodology 400).

FIG. 3 is a diagram illustrating exemplary methodology 300 for forming the present CNT-based hydrocarbon detectors. As provided above, in this example, the amphiphilic coating is formed on the CNTs by reacting (covalently bonding) an amphiphilic polymer with the surface of the CNTs. An exemplary coating that may be produced in accordance with methodology 300 is depicted in FIG. 5 (described below).

In step 302, a solution is prepared containing the amphiphilic polymer molecules (used to form the CNT coating) or precursors thereof solubilized in a solvent (such as water). Suitable polymer precursors include any compounds containing a polymer initiator, such as a molecule carrying an insaturation such as a double bond or triple bond, or a molecule with a nucleophile functional group, such as an amine or an alcohol.

According to an exemplary embodiment, the amphiphilic molecules contain a hydrophobic moiety selected from the group consisting of an alkyl chain or a hydrophobic polymer (such as polyethylene or polypropylene), and a hydrophilic moiety selected from the group consisting of a hydroxamic acid, a hydroxamate, a carboxylic acid, a carboxylate, a sulfonic acid, a sulfonate, a phosphonic acid and a phosphonate. It is notable that very high functionalization densities on the CNT surfaces of up to 1 functional group per 6 carbons can be obtained when using in-situ generated diazonium salts of the corresponding precursor, which is preferably an amine. The diazonium salt moiety facilitates attachment of the amphiphilic molecules to the surface of the CNTs. For a general discussion of in-situ formation of diazonium salts and carbon nanotube functionalization see, for example, Bahr et al., “Highly Functionalized Carbon Nanotubes Using in Situ Generated Diazonium Compounds,” J. Am. Chem. Soc., Chem. Mater. 2001, 13, 3823-3824, the entire contents of which are incorporated by reference herein.

In step 304, CNTs are added to the solution prepared in step 302. Commercially available CNTs may be used in the preparation. Due to the amphiphilic nature of the molecules, the molecules will assemble on, and interact with (via covalent bond, e.g., via the diazonium salt moiety), the surfaces of the CNTs (forming an amphiphilic coating) with the hydrophilic moieties facing outward, and the hydrophobic moieties forming a hydrophobic environment proximal to the surface of the CNTs (e.g., a distance of one to several carbon-carbon covalent bonds from the surface of the CNTs). It is in this hydrophobic environment where the active molecules will be sequestered. The result of step 304 is a dispersion of coated CNTs in the solvent (e.g., water).

In one exemplary embodiment, the amphiphilic molecule employed in accordance with methodology 300 is a hydroxamic acid (HA)-containing polymer. The resulting CNT coating in that case (i.e., HA-CNTs) is depicted in FIG. 5. The hydrophobic and hydrophilic parts of the coating are shown labeled in FIG. 5.

Referring back to FIG. 3, in step 306, small active, hydrophobic molecules are introduced to the hydrophobic environment created by the amphiphilic coating proximal to the surface of the CNTs. Based on steps 302 and 304, the coated CNTs are present in an (e.g., aqueous) solution. By way of example only, the active molecules can be introduced into the CNT coating (i.e., into the hydrophobic environment proximate to the surface of the CNTs created by the coating) by simply adding the active molecules to the solution.

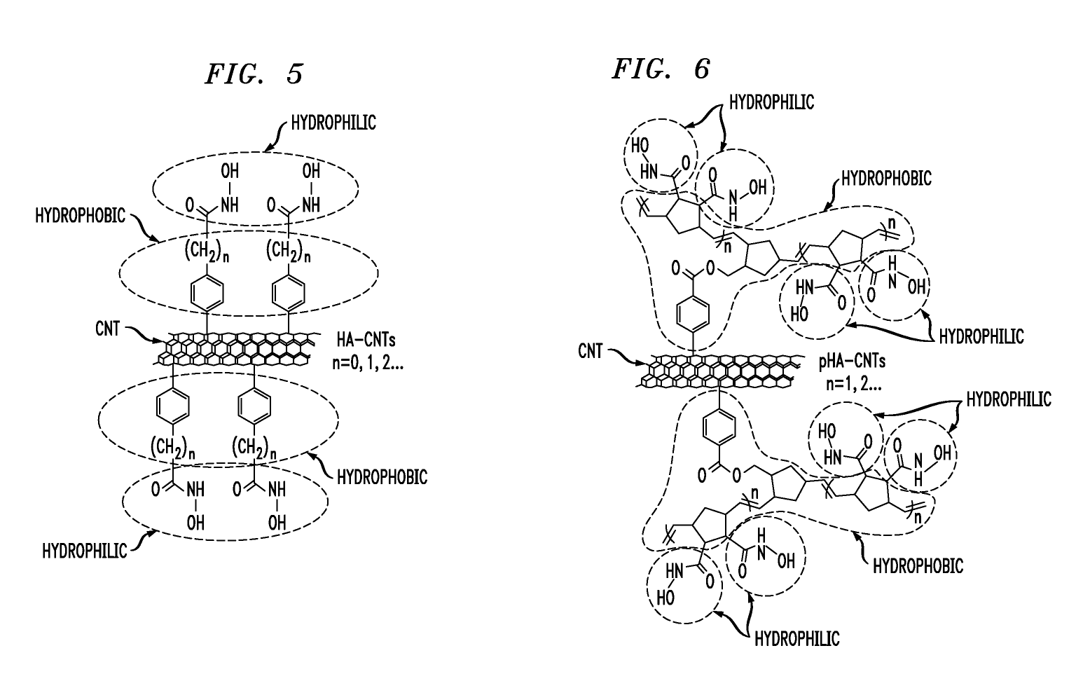

Namely, CNTs bearing hydroxamic acid-functionalized coatings (e.g., HA-CNTs (FIG. 5) and pHA-CNTs (FIG. 6, described below)) for instance have very high solubilities in water of up to several milligrams per milliliter. See, for example, Tulevski et al., “Chemically Assisted Directed Assembly of Carbon Nanotubes for the Fabrication of Large-Scale Device Arrays,” J. AM. CHEM. SOC., 129, 11964 (September 2007), the entire contents of which are incorporated by reference herein. When a hydrophobic small organic molecule is put in contact with a dispersion of HA-CNTs or pHA-CNTs in water, the hydrophobic part of the CNT coating absorbs the small molecule, since this hydrophobic interaction is preferred over interaction with the polar solvent.

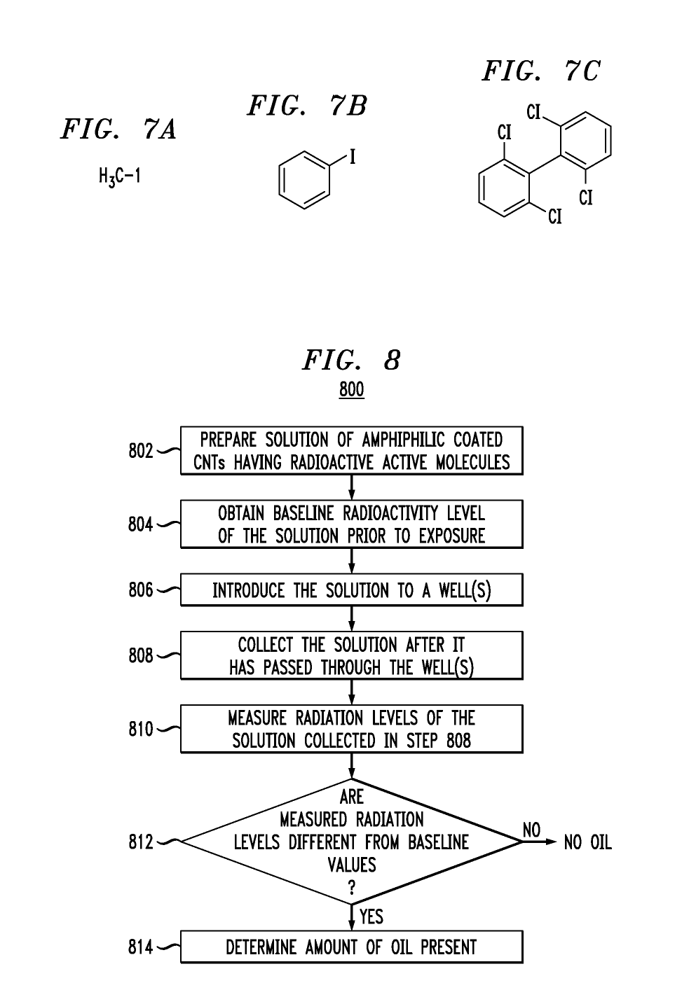

A small molecule to be used for this purpose might be a hydrophobic molecule capable of exhibiting radioactivity, such as iodomethane, iodobenzene, or tetrachlorobiphenylene labeled with radioactive carbon or iodine isotopes. See FIGS. 7A-C, which illustrate iodomethane, iodobenzene, and tetrachlorobiphenylene molecules, respectively.

An alternatively exemplary methodology 400 is provided in FIG. 4 for forming the present CNT-based hydrocarbon detectors. As provided above, in this example, the amphiphilic coating is formed on the CNTs by growing a polymer off the surface of the CNTs. An exemplary coating that may be produced in accordance with methodology 400 is the “polymer brush” coating depicted in FIG. 6 (described below).

In step 402, a solution is prepared containing a polymer precursor solubilized in a solvent (such as water). As provided above, suitable polymer precursors include any compounds containing a polymer initiator, such as a molecule carrying an insaturation such as a double bond or triple bond, or a molecule with a nucleophile functional group, such as an amine or an alcohol. As provided above, very high functionalization densities on the CNT surfaces can be achieved by using in-situ generated diazonium salts of the corresponding precursor, which facilitates attachment of the polymer precursors to the surfaces of the CNTs.

According to one exemplary embodiment, a polymer precursor is employed (any of the above-stated polymer precursors would suffice) that can be subsequently converted into a polymer brush bearing many hydroxamic acid functionalities (pHA). See description of FIG. 6, below.

In step 404, CNTs are added to the solution prepared in step 402. Commercially available CNTs may be used in the preparation. By way of step 404, the polymer precursors will interact (covalently, e.g., via the diazonium salt moiety) with the surfaces of the CNTs resulting in CNT surfaces modified with the polymer precursor. As above, the goal here is as well to form an amphiphilic coating on the surfaces of the CNTs wherein the hydrophobic parts of the coating form a hydrophobic environment proximal to the surface of the CNTs (e.g., a distance of one to several carbon-carbon covalent bonds from the surface of the CNTs). It is in this hydrophobic environment where the active molecules will be sequestered. The result of step 404 is a dispersion of polymer precursor coated CNTs in the solvent (e.g., water). An advantage to methodology 400 where the amphiphilic molecules are grown on the CNTs (templated from the polymer precursors) is that a potentially greater amount of surface functionality can be achieved, and thus leading to a greater sensitivity of the detectors.

Next, amphiphilic polymer molecules are grown on the surface of the CNTs off of the polymer precursors on the surfaces of the CNTs. Specifically, in step 406, a monomer is added to the dispersion (from step 404) to form/grow a polymer on the surfaces of the CNTs. By way of example only, suitable monomers employed in this step can include, but are not limited to, norbornene-based monomers, cyclic esters and ethers, or molecules carrying an insaturation such as a double bond or triple bond. By way of this process outlined in steps 404 and 406, for instance, polymer brushes—such as those shown in FIG. 6, can be grown on the surfaces of the CNTs. The polymer molecules grown on the surfaces of the CNTs forms the amphiphilic coating on the surfaces of the CNTs. Specifically, the polymers formed in this step include both a hydrophobic part and a hydrophilic part (see, for example, FIG. 6). The resulting coating thus forms a hydrophobic environment proximal to the surfaces of the CNTs in which the active molecules will be sequestered.

Namely, in one exemplary embodiment, the amphiphilic molecule employed in accordance with methodology 400 is a polymer brush bearing many hydroxamic acid functionalities (pHA). The resulting CNT coating in that case (i.e., pHA-CNTs) is depicted in FIG. 6. The hydrophobic and hydrophilic parts of the coating are shown labeled in FIG. 6. For a general description of polymer brushes see, for example, O. Azzaroni “Polymer Brushes here, there, and everywhere: Recent advances in their practical application and emerging opportunities in multiple research fields,” Journal of Polymer Science, Part A: Polymer Chemistry, vol. 50, issue 16, pages 3225-3258 15 Aug. 2012 (published May 2012), the entire contents of which are incorporated by reference herein. As highlighted above, an advantage of the coating configuration shown in FIG. 6 (as opposed for example to that shown in FIG. 5) is that a thicker coating can be obtained around the CNTs, and many more hydroxamic acid functionalities can be attached this way.

Referring back to FIG. 4, in step 408, small active, hydrophobic molecules are then introduced to the hydrophobic environment created by the amphiphilic coating proximal to the surface of the CNTs. Based on steps 402-406, the coated CNTs are present in an (e.g., aqueous) solution. By way of example only, the active molecules can be introduced into the CNT coating (i.e., into the hydrophobic environment proximate to the surface of the CNTs created by the coating) by simply adding the active molecules to the solution.

Namely, CNTs bearing hydroxamic acid-functionalized coatings (e.g., HA-CNTs or pHA-CNTs) for instance have very high solubilities in water of up to several milligrams per milliliter. When a hydrophobic small organic molecule is put in contact with a dispersion of HA-CNTs or pHA-CNTs in water, the hydrophobic part of the CNT coating absorbs the small molecule, since this hydrophobic interaction is preferred over interaction with the polar solvent.

As provided above, suitable small molecule include, but are not limited to, a hydrophobic molecule capable of exhibiting radioactivity, such as iodomethane, iodobenzene, or tetrachlorobiphenylene labeled with radioactive carbon or iodine isotopes. See FIGS. 7A-C.

FIG. 8 is a diagram illustrating an exemplary implementation of the above-described techniques to detect the presence of underground oil. In step 802, a solution is prepared containing the coated CNTs having radioactive-labeled (hydrophobic) organic molecules sequestered in the hydrophobic environment created proximal to the surface of the CNTs (see above). In the example, the coated CNTs are dispersed in water (which is facilitated by the hydrophilic part of the coating. In an exemplary embodiment, step 802 is carried out in accordance with either methodology 300 of FIG. 3 or methodology 400 of FIG. 4 (see above).

In step 804, a base line measurement of the level of radioactivity of the coated CNTs in the solution is preferably taken. This permits the later analysis of radioactivity levels against an initial reading once the solution has been introduced into a well. Namely, as described above, the amount of active molecules which leach from the coating is proportional to the amount of hydrophobic material (in this case oil) to which the CNTs have been exposed. Thus having an initial reading (prior to exposure of the coated CNTs to oil) would be helpful. Any suitable radiation detection device that can quantify a level of radiation can be implemented in accordance with the present techniques. By way of example only, a suitable radiation detection device is described in U.S. patent application Ser. No. 13/955,740, filed by Afzali-Ardakani et al., entitled “Radiation Detector Based on Charged Self-Assembled Monolayers on Nanowire Devices,” the entire contents of which are incorporated by reference herein.

According to an exemplary embodiment, the process involves creating one or more wells in the soil in areas where the detection of oil is desired. The size, depth, location, etc. of the wells is application specific and within the capabilities of one skilled in the art to determine but generally it can have a diameter of from a few inches to several hundreds of inches.

In step 806, the solution is introduced into the well. For instance, the solution can be poured or sprayed into the well, which can expose the solution to strata of earth through which the well passes, as well as the bottom of the well. The amount of solution inserted into the well depends on the diameter and the depth of the well. In a general scenario, the amount of the solution introduced into the well can be anywhere from a few liters to hundreds of gallons depending on the dimensions of the well. Upon exposure to hydrophobic material (such as oil) in the well, the active molecules will come out from the hydrophobic region of the coating and leach into the soil. Again, the amount of the active molecules that are transferred from the coated CNTs to the soil surrounding the wells and/or to the oil reservoir is proportional to the amount of hydrocarbon/oil to which the coated CNTs are exposed.

After passing through the well, in step 808, the solution is collected, i.e., extracted (or in some other manner retrieved), from the well. By way of example only, tubing, piping, or some other suitable conduit can be used to draw the solution up from the bottom of the well. Alternatively, the well can be formed having an (input) opening and an (output) opening. For example, a u-shaped well can be formed with both ends of the well opening to the surface. In that case, the solution can be flowed into the well via the input and retrieved from the well via the output. The radiation of the solution flowing out of the well (from the output) can be measured (e.g., as per methodology 800 of FIG. 8, described below).

In step 810, the radiation levels of the solution collected (in step 808) from the well are measured. The notion here is that if there is hydrocarbon material (e.g., oil) in the well, then the (radio-labeled) molecules which leach into the soil will lower the radioactivity of the solution collected (post exposure) from the well. Thus, a lower radiation reading of the post-exposure solution (collected in step 808) vis-à-vis the baseline readings (from step 804) would indicate exposure to hydrocarbon materials.

Specifically, the radioactivity reading(s) from step 810 can be analyzed by way of comparison with the base line radioactivity measurements from step 804. Namely, in step 812, a determination is made as to whether the radioactivity readings from steps 804 (initial) and 810 (after passing through the well) are different. If the radioactivity readings from steps 804 (initial) and 810 (after passing through the well) are the same (i.e., differ by less than about 10%), then it can be assumed that the coated CNTs were not exposed to oil or any other kind of hydrocarbon material in the well, and thus there is no oil in the well. On the other hand, if the radioactivity readings from steps 804 (initial) and 810 (after passing through the well) are different (i.e., differ by more than about 10%), then it can be assumed that the coated CNTs were exposed to oil in the well, and thus there is oil in the well.

In that case, a determination may be made in step 814 as to an amount of the oil in the well. Again, as above, the amount of the active molecules that are transferred from the coated CNTs to the soil is proportional to the amount of hydrocarbon/oil to which the coated CNTs are exposed. This calculation can be conducted in a number of different ways. For instance, in one exemplary embodiment, step 814 is performed to determine in a relative sense which wells have more or less oil than other wells. Basically, multiple wells are treated (as described above), and the radioactivity level readings from each of the treated wells are compared to determine which of the wells has/have more oil than others.

Alternatively, a more quantitative analysis can be performed, for instance, by comparing the measured radiation levels from the solution collected from the well(s) with known (calibrated) radiation levels based on known levels of hydrocarbon exposure. For example, a control solution prepared as described, for example, in conjunction with the description of FIG. 3 or FIG. 4, above can also be made and exposed to known amounts of hydrocarbon. The amounts of hydrocarbon used for the control can be varied to increase/decrease the sensitivity of the test (and would be within the capabilities of one skilled in the art to determine for a given application). The hydrocarbon can be removed (and along with it at least a portion of the active molecules). The radioactivity levels can then be measured in this manner for varying amounts of hydrocarbon exposure to attain a spectrum of (known) exposure values. Then, when readings are taken at a given well (i.e., with the same solution from FIG. 3/FIG. 4), these (radioactivity) readings can be compared with the known values to determine the amount of hydrocarbon exposure.

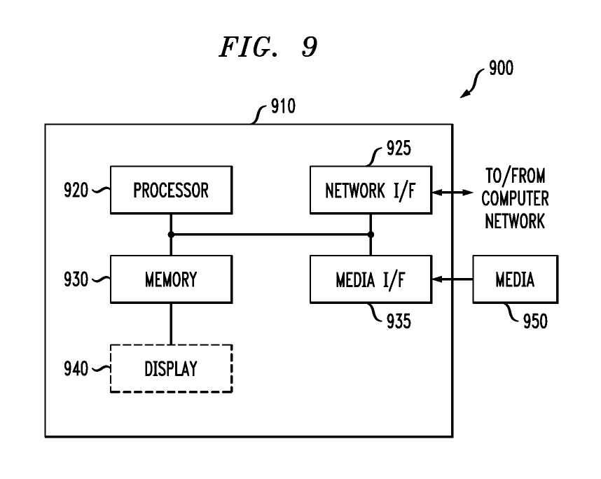

The computational steps of methodology 800 may be performed by an apparatus, such as apparatus 900 of FIG. 9. Namely, FIG. 9 is a block diagram of an apparatus 900 for implementing one or more of the methodologies presented herein. By way of example only, apparatus 900 can be configured to implement one or more of the steps of methodology 800 of FIG. 8 to detect the presence of underground oil.

Apparatus 900 comprises a computer system 910 and removable media 950. Computer system 910 comprises a processor device 920, a network interface 925, a memory 930, a media interface 935 and an optional display 940. Network interface 925 allows computer system 910 to connect to a network, while media interface 935 allows computer system 910 to interact with media, such as a hard drive or removable media 950.

As is known in the art, the methods and apparatus discussed herein may be distributed as an article of manufacture that itself comprises a machine-readable medium containing one or more programs which when executed implement embodiments of the present invention.

The machine-readable medium may be a recordable medium (e.g., floppy disks, hard drive, optical disks such as removable media 950, or memory cards) or may be a transmission medium (e.g., a network comprising fiber-optics, the world-wide web, cables, or a wireless channel using time-division multiple access, code-division multiple access, or other radio-frequency channel). Any medium known or developed that can store information suitable for use with a computer system may be used.

Processor device 920 can be configured to implement the methods, steps, and functions disclosed herein. The memory 930 could be distributed or local and the processor device 920 could be distributed or singular. The memory 930 could be implemented as an electrical, magnetic or optical memory, or any combination of these or other types of storage devices. Moreover, the term “memory” should be construed broadly enough to encompass any information able to be read from, or written to, an address in the addressable space accessed by processor device 920. With this definition, information on a network, accessible through network interface 925, is still within memory 930 because the processor device 920 can retrieve the information from the network. It should be noted that each distributed processor that makes up processor device 920 generally contains its own addressable memory space. It should also be noted that some or all of computer system 910 can be incorporated into an application-specific or general-use integrated circuit.

Optional display 940 is any type of display suitable for interacting with a human user of apparatus 900. Generally, display 940 is a computer monitor or other similar display.

Although illustrative embodiments of the present invention have been described herein, it is to be understood that the invention is not limited to those precise embodiments, and that various other changes and modifications may be made by one skilled in the art without departing from the scope of the invention.

What is claimed is:

1. A hydrocarbon detection device, comprising:
a plurality of carbon nanotubes dispersed in a polar solvent, wherein each of the carbon nanotubes is coated with an amphiphilic coating comprising molecules with a hydrophilic moiety and a hydrophobic moiety, and wherein the coating creates a hydrophobic environment proximal to a surface of each of the carbon nanotubes; and
one or more hydrophobic active molecules sequestered in the hydrophobic environment proximal to the surface of each of the carbon nanotubes.

2. The hydrocarbon detector of claim 1, wherein the polar solvent is water.

3. The hydrocarbon detector of claim 1, wherein the hydrophilic moiety is selected from the group consisting of: a hydroxamic acid, a hydroxamate, a carboxylic acid, a carboxylate, a sulfonic acid, a sulfonate, a phosphonic acid, and a phosphonate.

4. The hydrocarbon detector of claim 1, wherein the hydrophobic moiety is selected from the group consisting of: an alkyl chain and a hydrophobic polymer.

5. The hydrocarbon detector of claim 1, wherein the active molecules are selected from the group consisting of: iodomethane, iodobenzene, and tetrachlorobiphenylene.

6. The hydrocarbon detector of claim 1, wherein the active molecules are labeled with a radioactive isotope.

7. A method of forming a hydrocarbon detection device, comprising the steps of:
(a) contacting carbon nanotubes with a solution comprising molecules with a hydrophilic moiety and a hydrophobic moiety solubilized in a polar solvent, wherein by way of the contacting step (a) the molecules interact with surfaces of the carbon nanotubes to form an amphiphilic coating on the surfaces of the carbon nanotubes which creates a hydrophobic environment proximal to a surface of each of the carbon nanotubes; and
(b) contacting the solution with one or more hydrophobic active molecules, wherein by way of the contacting step (b) the hydrophobic active molecules are sequestered in the hydrophobic environment proximal to the surface of each of the carbon nanotubes.

8. The method of claim 7, wherein the polar solvent is water.

9. The method of claim 7, wherein the hydrophilic moiety is selected from the group consisting of a hydroxamic acid, a hydroxamate, a carboxylic acid, a carboxylate, a sulfonic acid, a sulfonate, a phosphonic acid, and a phosphonate.

10. The method of claim 7, wherein the hydrophobic moiety is selected from the group consisting of: an alkyl chain and a hydrophobic polymer.

11. The method of claim 7, wherein the active molecules are selected from the group consisting of: iodomethane, iodobenzene, and tetrachlorobiphenylene.

12. The method of claim 7, wherein the active molecules are labeled with a radioactive isotope.

13. A method of forming a hydrocarbon detection device, comprising the steps of:
(a) contacting carbon nanotubes with a solution comprising polymer precursors solubilized in a polar solvent, wherein by way of the contacting step (a) the polymer precursors interact with surfaces of the carbon nanotubes, modifying the surfaces of the carbon nanotubes with the polymer precursors;
(b) adding monomers to the solution, wherein by way of the adding step (b), the monomers will interact with the polymer precursors on the surfaces of the carbon nanotubes to form polymers on the surfaces of the carbon nanotubes with a hydrophilic moiety and a hydrophobic moiety, and wherein the polymers form an amphiphilic coating on the surfaces of the carbon nanotubes which creates a hydrophobic environment proximal to a surface of each of the carbon nanotubes; and
(c) contacting the solution with one or more hydrophobic active molecules, wherein by way of the contacting step (c) the hydrophobic active molecules become sequestered in the hydrophobic environment proximal to the surface of each of the carbon nanotubes.

14. A method of hydrocarbon detection, comprising the steps of:
preparing a solution comprising i) a plurality of carbon nanotubes dispersed in a polar solvent, wherein each of the carbon nanotubes is coated with an amphiphilic coating comprising molecules with a hydrophilic moiety and a hydrophobic moiety, and wherein the coating creates a hydrophobic environment proximal to a surface of each of the carbon nanotubes, and ii) one or more hydrophobic active molecules sequestered in the hydrophobic environment proximal to the surface of each of the carbon nanotubes wherein the active molecules are labeled with a radioactive isotope;
introducing the solution into one or more oil wells such that upon any exposure to hydrocarbons in the wells one or more of the hydrophobic active molecules are transferred from the hydrophobic environment proximal to the surface of the carbon nanotubes to the hydrocarbons;
collecting the solution once the solution has passed through the wells; and
analyzing radioactivity levels of the solution collected from the wells.

15. The method of claim 14, wherein the polar solvent is water.

16. The method of claim 14, wherein the hydrophilic moiety is selected from the group consisting of: a hydroxamic acid, a hydroxamate, a carboxylic acid, a carboxylate, a sulfonic acid, a sulfonate, a phosphonic acid, and a phosphonate.

17. The method of claim 14, wherein the hydrophobic moiety is selected from the group consisting of: an alkyl chain and a hydrophobic polymer.

18. The method of claim 14, wherein the active molecules are selected from the group consisting of: iodomethane, iodobenzene, and tetrachlorobiphenylene.

19. The method of claim 14, further comprising the step of:
determining a baseline radioactivity level of the solution prior to introducing the solution into the wells.

20. The method of claim 19, further comprising the steps of:
measuring radioactivity levels from the solution collected from the wells; and
comparing the radioactivity levels of the solution collected from the wells to determine whether hydrocarbons are present in any of the wells.

21. The method of claim 14, further comprising the steps of:
measuring radioactivity levels of the solution collected from the wells; and
determining an amount of hydrocarbons present in the wells based on the radioactivity levels of the solution collected from the wells, wherein the radioactivity levels are based on an amount of the hydrophobic active molecules which are transferred from the hydrophobic environment proximal to the surface of the carbon nanotubes to the soil surrounding the wells.
